(19) United States
(12) Patent Application Publication
(10) Pub. No.: US 2001/0041942 A1
(43) Pub. Date: Nov. 15, 2001

(54) Novel composite and its use

(76) Inventors: Heimo Ylanen, Abo (FI); Hannu Aro, Turku (FI); Kaj Karlsson, Turku (FI); Antti Yli-Urpo, Littoinen (FI); Mikko Hupa, Turku (FI); Egon Nordstrom, Pargas (FI)

Correspondence Address: James C. Lydon, Attorney at Law, Suite 100, 100 Daingerfield Road, Alexandria, VA, 22314, US

(21) Appl. No.: 09/875,018

(22) Filed: Jun. 7, 2001

Related U.S. Application Data
(63) Continuation of application No. PCT/FI99/00960, filed on Nov. 19, 1999.

(30) Foreign Application Priority Data
Dec. 11, 1998 (FI) 982682

(51) Int. Cl. A61F 2/02; A61F 2/28; A61C 8/00

(52) U.S. Cl. 623/23.76; 433/201.1; 623/23.5; 623/23.51; 623/23.57

(57) ABSTRACT

The invention relates to a porous composite which comprises particles made from a bioactive material, the particles being sintered together to form a porous composite. It is characteristic that the particles have one or more recesses or throughgoing holes, or that the particles provided with an unbroken surface layer are hollow.

The invention relates to a porous composite as defined in claim 1. The invention further concerns an implant the surface of which is partly covered with the said composite.

### BACKGROUND OF THE INVENTION AND STATE OF THE ART

The publications to which reference is made below and which are used for illustrating the background of the invention and the state of the art are to be deemed as being incorporated into the description of the invention below.

### Biomaterials and Their Biologic Attachment

Implants for both medical and dental purposes have long been prepared from a variety of materials. Various metals, metal alloys, plastics, ceramic materials, glass ceramic materials, and the latest, i.e. bioactive glasses, differ one from another not only by their durability but also by the properties of the interface between the implant and the tissue. Inert materials, such as metals and plastics, do not react with a tissue, in which case there always remains an interface between the implant and the tissue; the implant and the tissue constitute two distinct systems. Bioactive materials, such as hydroxyapatite, glass ceramic materials and bioactive glasses, react chemically with the tissue, whereupon there forms at the interface between the implant and the tissue a chemical bond, which is relatively strong, especially with bioactive glasses. The implant and the tissue are thus fixed to each other. The speed of the healing of the tissue and the possible chemical bond with the implant depend on the tissue activity of the implant material used.

In the planning of the interface of the implant it should additionally be taken into consideration that implants intended for functional activity are subjected to motion under a load immediately after the surgery. This hampers healing and impairs the final result. Furthermore, the structure of a rigid implant does not transmit the load to the resilient bone; the interfacial region concerned is disturbed and integration is hindered. Problems are often also caused by paucity of the bone or its inferior quality. If, for example, a dental implant is placed surgically in scarce or low-quality bone, initial stability is not attained and the operation will fail if bone is not generated in advance. In the functional conditions cited above, undisturbed healing cannot be achieved with conventional implants.

### Specific Clinical Problems Associated with Implants

1. Mechanical micromotion between the implant and the host tissue hinders their rapid integration (osseous bond) within 6-12 weeks, in which case the piece remains without permanent firm attachment to the surrounding tissue. It is known that this lack of an osseous bond will lead to slow clinical detachment of the implant at an early stage (within 1-2 years) or even years later, and to a need for repeat surgery.

2. One method is to make the implant surface porous, for example, by means of a three-dimensional surface structure a few millimeters thick constructed from microscopic titanium spheres or titanium tape. New bone from the host tissue is expected to grow into this surface structure. Such a porous, biologically inactive surface structure will produce a microscopic locking structure for the ingrowing new bone, but the mechanical properties of this attachment are not capable of adapting sufficiently to the load conditions. In an optimal structure of an osseous bond between an implant and the host tissue there occurs continuous readaptation, the purpose of which is to adapt the strength of the structure to correspond to the load conditions.

3. It has been shown that the attachment of a metallic bone implant (such as an artificial joint) to the host bone can be promoted by means of a bioactive coating. The most commonly used material is synthetic hydroxyapatite. It has been found that hydroxyapatite 1) promotes the mechanical attachment to the host bone of a bone implant which has been attached firmly by surgery and 2) reduces the interference caused by micromotion in the attachment of a bone implant to the host bone and 3) reduces the retardation caused by local lack of bone or the lack of contact to the bone implant in the integration of the implant. Hydroxyapatite is attached to the implant surface by a spraying technique, in which case the coating material is mainly applied to the open surface only from the spraying direction. The biomechanically and biologically most optimal implant surface forms a 3-dimensional structure, wherein the interstitial space of the structure forms a growth space for the ingrowing bone tissue. Healing in this case leads to the formation of a connecting microscopic locking structure. New tissue growth is induced if the porous structure is made completely of a bioactive material. In this case the bioactive coating material forms a 3-dimensional osteoconductive surface for new bone growth. In exceptionally difficult conditions, in which the growth of the host bone is especially poor, for example owing to the poor quality or paucity of the bone, new bone growth can possibly be induced by combining with the bioactive coating material an osteoinductive component which directly induces bone formation.

Even though the bioactive coating may improve the integration of the implant to the host bone, it is to be noted, however, that there are a number of problems associated with this technique. The combination of two materials differing in their properties (elasticity, thermal expansion) is technically demanding. The coating of a metal implant with a bioactive ceramic material may lead to early breakdown of the coating, its rapid corrosion or its slow detachment (delamination). This has proven to be the most common complication in attempts to use bioceramic materials, including hydroxyapatite, as a smooth coating material of metallic implants.

One further problem involved with implants provided with prior-art bioactive coatings is that the bioactive surface, which is rather brittle, is easily damaged when the implant is chased into the bone.

International patent publication WO 98/47465, Ylänen et al., describes an implant which allows micromotion between the implant and the surrounding tissue (bone) while, nevertheless, ensuring rapid integration of the implant and the bone. The said implant can be chased into bone without a risk of the bioactive coating being damaged. The implant is made up of a body and a bioactive layer which covers only a portion of the implant surface. In the frame of the implant there is a recess or a throughgoing hole, which contains a porous composite comprising bioactive particles, the composite forming the surface layer of the implant only in the area of the recess or the throughgoing hole.

The same patent publication also describes a new porous composite suitable for the above-mentioned purpose, the composite comprising i) particles A made of a bioactive material and ii) particles B, which are made of a non-bioactive or weakly bioactive material sintrable to the said bioactive material. The said particles A and particles B are sintered together to form a porous composite. Combined with the implant, the said composite ensures both rapid ossification and permanent attachment of the implant.

International patent publication WO 96/21628, Brink et al., describes a group of bioactive glasses which can be processed easily. From such bioactive glasses it is possible, for example to draw fibers and, for example by the torch spraying technique, to prepare so-called microspheres of glass. In the above-mentioned composite, such microspheres have been used as the bioactive particles. Porous bioactive pieces are prepared by sintering these microspheres together. By using microspheres which are within as narrow a fraction as possible (of as uniform a size as possible), it is possible to control the porosity of the body. According to the literature it seems that the most advantageous particle size is within the fraction 200-400 microns (Schepers et al. 1997, Tsuruga et al. 1997, Schliephake et al. 1991, Higashi et al. 1996). The studies carried out by the inventors so far have shown that a porous bioactive implant which has been prepared by sintering bioactive microspheres of the fraction 250-300 microns reacts very strongly in the femur of a rabbit (Ylänen et al. 1997). The results of the studies have shown that the said implant model reacts rapidly and the porous matrix fills at a steady speed with new bone. The shear strength of the bioactive implants in a push-out to failure test has been already after three weeks statistically as high as after 12 weeks. The amount of bone inside the matrix has been after 12 weeks 35-40% of the pore volume both in bioactive implants and in the titanium implants used as controls. It is, however, advisable to note that in a bioactive matrix porosity increases evenly as a function of time as the bioactive glass mass decreases. Porosity increased in experiments in vivo from 30% to 65%. The porosity of titanium, of course, does not change in any way. Thus the amount of new bone inside bioactive implants is defacto almost double that inside titanium implants. In our opinion this shows that the porous implant type used by us is right.

The beginning of new bone growth seems to be located in micro-cracks in the bioactive glass particles (Schepers et al. 1997). Evidently the calcium and phosphate dissolving from the glass into the fluid (in vitro SBF, in vivo plasma) surrounding the micro-crack form, together with the calcium and phosphate normally in the fluid, so high a concentration that the solubility product of the ions concerned is exceeded. As a consequence of this, calcium phosphate precipitates onto the silica gel on the surface of the bioactive glass and new bone growth begins. The porous body sintered from bioactive microspheres is full of microscopically small cavities. This explains the rapid bone growth inducing property of the bodies we sintered from bioactive microspheres. It has further been shown that the roughness of the surface has a favorable effect on the attachment to the biomaterial surface of proteins which control bone growth (Grossner et al. 1991, Boyan et al. 1998), as well as has the biomaterial itself. According to the literature, the said proteins attach best and most rapidly to the surface of bioactive glass (Ohgushi et al. 1993, Vrouwenvelder et al. 1992, Lobel et al. 1998, Vrouwenvelder et al. 1993, Shimizu et a. 1997, Miller et al. 1991).

However, the composite described in patent publication WO 98/47465, which is made up of smooth glass spheres having an untreated surface, must be in body fluid contact for about a week before the silica gel layer required by bone growth is formed on the surface of the spheres. Only after this period can the actual bone formation begin.

### OBJECT OF THE INVENTION

It is an object of the invention to provide a new bioactive and porous composite which, combined with an implant, will ensure more rapid ossification than do prior-art composites.

It is a particular object of the invention to provide a bioactive porous composite on the surface of which there is already a bioactive layer required for the induction of bone growth, in which case the integration of the bone to the composite can begin immediately after the composite comes into contact with the body fluid, i.e. immediately after the surgery.

### SUMMARY OF THE INVENTION

The characteristics of the invention are given in the independent claims.

The invention thus relates to a porous composite which comprises particles made of a bioactive material, the particles being sintered together to form a porous composite. It is characteristic that the particles have one or more recesses or throughgoing holes, or that the particles provided with an unbroken surface layer are hollow.

The invention additionally relates to an implant which is made up of a body and a bioactive layer extending to the surface of the implant and covering only a portion of the implant surface. In the body of the implant there is a recess or a throughgoing hole which contains the composite comprising particles which are made of a bioactive material and are sintered together, the composite forming a layer which extends to the surface of the implant only in the area of the recess or the throughgoing hole. It is characteristic that the composite is the composite according to the present invention.

### BRIEF DESCRIPTION OF THE FIGURES

FIG. 1 depicts a hip prosthesis having three recesses for the composite according to the invention, and

FIG. 2 depicts a cross section of a recess made in the implant body and the composite according to the invention placed in it.

### PREFERRED EMBODIMENTS OF THE INVENTION AND A DETAILED DESCRIPTION

### Definitions

By the term “implant” is meant in the present invention any body, made of a man-made material, to be placed in a tissue, such as an artificial joint or part thereof, a screw, a fixation plate, or a corresponding orthopedic or dental device.

In the context of the definition of the present invention, by “bioactive material” is meant a material which in physiological conditions dissolves at least partly in a few months, preferably within a few weeks, most preferably in approximately 6 weeks. The bioactive material may, for example, be a bioactive glass, a bioactive ceramic material or a bioactive glass ceramic material.

In the context of the definition of the present invention, the term “non-bioactive or weakly bioactive material” denotes a material which in physiological conditions does not dissolve within the first months. This material may be, for example, a non-bioactive or weakly bioactive glass, ceramic material, glass ceramic material or hydroxyapatite. This material may thus be any physiologically suitable material the bioactivity of which is clearly weaker than the material of the bioactive particles, and which additionally is such that the bioactive particles and the less or not at all bioactive particles can be sintered together to form a porous composite.

“Recess in a particle” denotes a recess made in a particle, the depth of the recess being typically several tens of microns, such as 50 microns or more. The topographic irregularities of the surface, produced by the roughening (etching) of the particles, are, on the other hand, typically in the order of magnitude of 1-50 microns.

### Especially Preferred Embodiments

Even before the particles are sintered there is made a recess or a throughgoing hole inside them. There may, of course be several recesses or holes in one and the same particle. According to one option, a particle which is hollow may be provided with an unbroken surface layer.

The surface of the particles forming the composite is preferably roughened by means of, for example, hydrogen fluoride vapor. The roughening can be carried out before the sintering or after it.

According to another embodiment, there is formed on the particle surfaces one or more bioactive layers, which are made up of, for example, silica gel and/or hydroxyapatite. Even though it is possible to form such bioactive layers on the surfaces of smooth particles, it is preferable that the surfaces of the particles are first roughened. Such preliminary corrosion, i.e. the formation of a bioactive layer, can be produced, for example, by using simulated body fluid (SBF) or some organic or inorganic solvent.

According to one preferred embodiment there is added to the bioactive layer some substance, typically a protein, such as a growth factor or the like, which induces bone growth.

Preferably the particles are of a substantially uniform size and mutually approximately of the same size.

The diameter of the particles is preferably within the range 100-500 μm, especially preferably within the range 200-400 μm.

According to one preferred embodiment, the particles are spherical, for example spheres prepared by the torch spraying technique, their raw material being bioactive glass.

According to another preferred embodiment, the particles are approximately cylindrical bodies. Such bodies may be prepared, for example, by drawing from glass a thin capillary tube which is cut into short pieces by using, for example, a carbon dioxide laser. In connection with the cutting, the capillary tube may become blocked at one or both ends. Thereby either a recess or a closed space is formed in the piece. In those pieces in which the capillary tube is not blocked, there forms a throughgoing hole.

A problem involved with many conventional bioactive glasses is that their processability is poor, because they crystallize easily. Spheres cannot be made from such bioactive glasses.

International patent application publication WO 96/21628 describes bioactive glasses of a novel type; their working range is suitable for the processing of glass and they can thus be used for making spheres and other bodies. The bioactive glasses described in this publication are especially good also for the reason that the processability of the glass has been achieved without the adding of aluminum oxide. Such glasses typically have the following composition:

SiO₂ 53-60% by weight

Na₂O 0-34% by weight

K₂O 1-20% by weight

MgO 0-5% by weight

CaO 5-25% by weight

B₂O₃ 0-4% by weight

P₂O₅ 0.5-6% by weight

however so that

Na₂O+K₂O=16-35% by weight,

K₂O+MgO=5-20% by weight and

MgO+CaO=10-25% by weight.

According to an especially preferred embodiment, the bioactive glass spheres or other bodies are made from bioactive glass the composition of which is Na₂O 6% by weight, K₂O 12% by weight, MgO 5% by weight, CaO 20% by weight, P₂O₅ 4% by weight and SiO₂ 53% by weight.

The composite may also comprise other particles, which are made from non-bioactive or weakly bioactive material sintrable with the said bioactive material. It is highly recommendable that the non-bioactive or weakly bioactive material should begin to dissolve before the bioactive material has dissolved completely.

Such “other particles” are suitably glass spheres made from a weakly bioactive glass, preferably glass having the composition Na₂O 6% by weight, K₂O 12% by weight, MgO 5% by weight, CaO 15% by weight, P₂O₅ 4% by weight, and SiO₂ 58% by weight.

The composite according to the invention may, of course, contain particles made from several bioactive materials and/or from several non-bioactive or weakly bioactive materials.

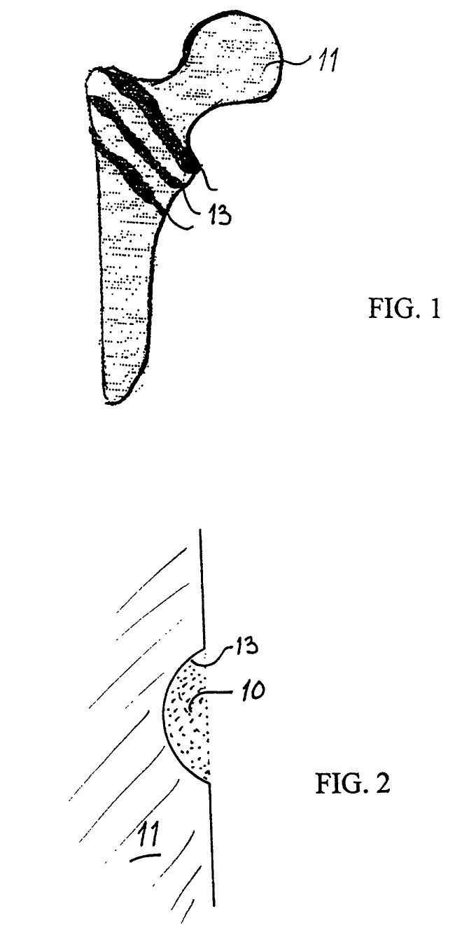

In an implant according to the present invention there is exploited the principle of noncontinuous coating, which is described in greater detail in publication WO 98/47465 mentioned above, and which is illustrated in accompanying FIGS. 1 and 2. In the implant body 11 there is made one or more recesses 13 or throughgoing holes (the latter option does not appear in the figures), and composite according to the invention is placed in such recesses or holes. Thus the composite will not cover the body surface entirely; the composite layer will form a layer 10 extending to the surface only in the area of the recess or recesses 13 (or the throughgoing hole/holes). FIG. 1 depicts a hip prosthesis having three ring-like recesses 13 which contain composite according to the invention. FIG. 2 depicts a cross section of an implant according to the invention; in the body 11 of the implant there is a recess 13 for the composite layer 10.

In the options of the figures it is possible, when so desired, to sinter also to the surface of the recess inert particles, suitably made from the body material, before the formation or addition of the composite into the recess.

According to one embodiment, the implant according to the invention can be prepared so that a composite in the recess (or throughgoing hole) is formed so that the particles are introduced into the recess, for example, mixed with a suitable organic binding agent. Thereafter, sintering is carried out, whereupon the organic binding agent burns.

According to another embodiment, at the sintering stage the composite may be formed into a piece of the desired shape and size, the piece being attachable to the recess or throughgoing hole in the implant body.

The sintered composite according to the invention is not only in the micro size (recesses/holes in the particles) but also in the macro size (the particles sintered together, either provided with recesses/holes or hollow, form a porous entity) full of independent islands favorable for new bone growth. The pre-roughened and pre-activated surface further speeds up the starting of reactions necessary for new bone formation.

The invention embodiments mentioned above are only examples of the implementation of the idea according to the invention. For a person skilled in the art it is clear that the various embodiments of the invention may vary within the framework of the claims presented below.

### Literature References

Schepers E J and Ducheyne P (1997) Bioactive glass particles of narrow size range for the treatment of oral bone defects: a 1-24 month experiment with several materials and particle sizes and size ranges. J Oral Rehabil, 24(3):171-181.

Tsuruga E, Takita H, Itoh H, Wakisaka Y and Kuboki Y (1997) Pore size of porous hydroxyapatite as the cell-substratum controls BMP-induced osteogenesis. J Biochem (Tokyo) 121(2):317-324.

Schliephake H, Neukam F W and Klosa D (1991) Influence of pore dimensions on bone ingrowth into porous hydroxylapatite blocks used as bone graft substitutes. A histometric study. Int J Oral Maxillofac Surg 20(1):53-58.

Higashi T and Okamoto H (1996) Influence of particle size of hydroxyapatite as a capping agent on cell proliferation of cultured fibroblasts. J Endod 22(5):236-239.

Ylänen H, Karlsson K H, Heikkila J T, Mattila K and Aro H T (1997) 10th International Symposium on Ceramics in Medicine, Paris.

Grossner-Schreiber B and Tuan R S (1991) The influence of the titanium implant surface on the process of osseointegration. Dtsch Zahnartzl Z 46(10):691-693.

Boyan B D, Batzer R, Kieswetter K, Liu Y, Cochran D L, Szmuckler-Moncler S, Dean D D and Schwartz Z (1998) Titanium surface roughness alters responsiveness of MG63 osteoblast-like cells to alpha, 25-(OH)2D3. J Biomed Mater Res 39(1):77-85.

Ohgushi H, Dohi Y, Tamai S and Tabata S (1993) Osteogenic differentiation of marrow stromal stem cells in porous hydroxyapatite ceramics. J Biomed Mater Res 27(11):1401-1407.

Vrouwenvelder W C, Groot C G and de Groot K (1992) Behaviour of fetal rat osteoblasts cultured in vitro on bioactive glass and nonreactive glasses. Biomaterials 13(6):382-392.

Lobel K D and Hench L L (1998) In vitro adsorbition and activity of enzymes on reaction layers of bioactive glass substrates. J Biomed Mater Res 39(4):575-579.

Vrouwenvelder W C, Groot C G and de Groot K (1993) Histological and biochemical evaluation of osteoblasts cultured on bioactive glass, hydroxylapatite, titanium alloy and stainless steel. J Biomed Mater Res 27(4):465-475.

Shimizu Y, Sugawara H, Furusawa T, Mizunuma K Inada K and Yamashita S (1997) Bone remodeling with resorbable bioactive glass and hydroxyapatite. Implant Dent 6(4):269-274.

Miller T A, Ishida K, Kobayashi M, Wollman J S, Turk A E and Holmes R E (1991) The induction of bone by an osteogenic protein and the conduction of bone by porous hydroxyapatite: a laboratory study in the rabbit. Plast Reconstr Surg 87(1):87-95.

1. A porous composite which comprises particles of a bioactive material which have been sintered together to form a porous composite, characterized in that the particles have one or more recesses or throughgoing holes or that particles provided with an unbroken surface layer are hollow.

1. A composite according to claim 1, characterized in that the particle surfaces are roughened.

3. A composite according to claim 1 or 2, characterized in that there are one or more bioactive layers formed on the particle surfaces.

4. A composite according to claim 3, characterized in that the layer is made up of silica gel and/or hydroxyapatite.

5. A composite according to claim 3 or 4, characterized in that a bone growth inducing substance has been added to the bioactive layer.

6. A composite according to any of the above claims, characterized in that the diameter of the particles is within the range 200-400 μm.

7. A composite according to any of the above claims, characterized in that the bioactive material forming the particles is a processable bioactive glass.

8. A composite according to claim 7, characterized in that the composition of the bioactive glass is Na₂O 6% by weight, K₂O 12% by weight, MgO 5% by weight, CaO 20% by weight, P₂O₅ 4% by weight, and SiO₂ 53% by weight.

9. A composite according to any of the above claims, characterized in that it also comprises other particles, which have been made from a non-bioactive or weakly bioactive material sintrable to the said bioactive material.

10. A composite according to claim 9, characterized in that the said other particles are made from a weakly bioactive glass, preferably a glass the composition of which is Na₂O 6% by weight, K₂O 12% by weight, MgO 5% by weight, CaO 15% by weight, P₂O₅ 4% by weight, and SiO₂ 58% by weight.

11. An implant which is made up of a body (11) and a bioactive layer (10) extending to the surface of the implant and covering only a portion of the implant surface, there being a recess (13) or a throughgoing hole in the implant body, the recess or hole containing a composite which comprises particles which have been made from a bioactive material and sintered together, the composite forming a layer (10) extending to the implant surface in the area of the recess (13) or throughgoing hole, characterized in that the composite is the composite of any of claims 1-10.

12. An implant according to claim 11, characterized in that the composite in the recess (13) or throughgoing hole has been formed so that the particles have been introduced into the recess or hole, whereafter the sintering has been carried out.

13. A composite according to any of claims 1-10, characterized in that it has been formed in the sintering stage into a piece of the desired shape and size which is attachable to the recess or throughgoing hole in the implant body.
